(19) United States
(12) Patent Application Publication
(10) Pub. No.: US 2026/0036047 A1
(43) Pub. Date: Feb. 5, 2026

(54) PROBE AND METHOD FOR SAMPLING FLUIDS FROM A SUBTERRANEAN ENVIRONMENT

(71) Applicant: Schlumberger Technology Corporation, Sugar Land, TX (US)

(72) Inventors: Deopaul Dindial, Dhahran (SA); Noor Ali Momin, Sugar Land, TX (US); Chen Tao, Sugar Land, TX (US); Ashers Partouche, Sugar Land, TX (US)

(21) Appl. No.: 19/266,305

(22) Filed: Jul. 11, 2025

Related U.S. Application Data
(60) Provisional application No. 63/678,224, filed on Aug. 1, 2024.

(51) Int. Cl. E21B 49/08; E21B 47/12; G01N 33/28

(52) U.S. Cl. CPC E21B 49/082; G01N 33/2823; E21B 47/12

(57) ABSTRACT

Embodiments presented provide for a probe for obtaining fluid samples from an underground environment. In embodiments, an arrangement is disclosed which has individually positionable probe blocks and inlets, with internal redundancy is provided for sampling at multiple locations with a single arrangement.

### CROSS-REFERENCE TO RELATED APPLICATIONS

This application claims the benefit of U.S. Provisional Application No. 63/678,224 filed Aug. 1, 2024.

### FIELD OF THE DISCLOSURE

Aspects of the disclosure relate to probes for testing. More specifically, aspects of the disclosure relate to a multi-function probe for testing fluid for hydrocarbon recovery operations.

### BACKGROUND

Oilfield service companies provide many services to enable recovery of hydrocarbons from geological stratum. To this end, sampling fluids from the subterranean environment is required from time to time to allow analysts to quantify and qualify the fluids downhole. Testing is very common in wellbores to allow the analysts to verify that the anticipated objectives can be met. If objectives cannot be met, then the wellbore may be reworked to correct identified problems. Since creation of wellbores can lead to an expected capital contribution of many millions of dollars, accurate placement and overall results are vital.

Sampling of fluids has taken place for decades in various forms. Starting in the early 2000's, different types of sampling technologies were developed to more accurately test more problematic wellbores. Problematic wellbores can have many challenges associated with them. One common problem is relatively unconsolidated fines that may be dislodged once a flow through a sample device is initiated. Once fluid flow is initiated by the testing apparatus, increasing flow into the testing apparatus will cause increased velocities of fluid flow in the geological stratum. After a specific breakaway velocity is achieved, fines that are stuck within the stratum dislodge and start to move. These fines transport with the fluid to the tester and, ultimately, lodge against the wellbore, a filter, or get stuck within the testing apparatus.

To deal with this problem, engineers for the testing apparatus provide a series of capabilities for the testing apparatus. Engineers install variable action pumping arrangements to allow for slower fluid draws from formations, to prevent breakaway fluid velocities from being achieved. More inventive and unique sample mechanisms are coupled with a packer system that allows the testing apparatus to seal to the wellbore more accurately. Filters may be installed at the entrance of the testing apparatus to prevent fines from entering the testing apparatus. In still more advanced systems, four sample points are provided, one sample point at 0 degrees, 90 degrees, 180 degrees, and 270 degrees to more accurately obtain fluid from the entire profile of the wellbore. Coupled with a packer system, such sampling systems can provide very good sampling; however, problems remain.

Some problematic wellbores are so unstable that the packer system associated with the testing apparatus cannot stabilize the wellbore for testing. In other instances, a perturbation in the wellbore will affect sampling along the axis of the wellbore. In such instances, sampling at one axial position is not fully representative of the entire wellbore. Moreover, conventional sampling devices do not provide for more than a single large packer. If setting problems exist in the wellbore, since there is a single packer setting device, operators are required to reposition and reset the entire device, thereby affecting overall efficiency. Such repositioning is common within the industry and requires operators to stop other operations to position the testing apparatus more accurately. Operators; therefore, need a testing apparatus that is more tolerant than conventional apparatus wherein testing can occur even if the operators overshoot their anticipated testing point.

There is a need to provide an apparatus and methods that are easier to operate than conventional apparatus and methods, wherein in problematic wellbores, more than one setting capability is provided.

There is a further need to provide apparatus and methods that do not have the drawbacks discussed above, namely sampling at a single point in a wellbore.

There is a still further need to reduce economic costs associated with operations and apparatus described above with conventional tools.

### SUMMARY

So that the manner in which the above recited features of the present disclosure can be understood in detail, a more particular description of the disclosure, briefly summarized below, may be had by reference to embodiments, some of which are illustrated in the drawings. It is to be noted that the drawings illustrate only typical embodiments of this disclosure and are; therefore, not to be considered limiting of its scope, for the disclosure may admit to other equally effective embodiments without specific recitation. Accordingly, the following summary provides just a few aspects of the description and should not be used to limit the described embodiments to a single concept.

In one embodiment, an arrangement is disclosed. The arrangement may comprise a body with a first end and a second end and an interface block. The arrangement may also comprise at least two first end inlets positioned in the first end and at least two second end inlets positioned in the second end, the first end inlets rotated related to the second end inlets. The arrangement may also comprise a first fluid system connected to the at least two first end inlets. The arrangement may also comprise a second fluid system connected to the at least two second end inlets. The arrangement may also comprise at least a first sample bottle connected to the first fluid end and at least a second sample bottle connected to the second fluid end, wherein the first fluid system is independent from the second fluid system.

In another example embodiment, a method for sampling fluids from a subterranean environment is disclosed. The method may comprise conveying a downhole apparatus to a location within a wellbore. The method may also comprise actuating at least one packer system in the downhole apparatus, the downhole apparatus having at least two inlets, each of the inlet rotated from the other inlet, wherein the downhole apparatus is configured with two independent packer systems. The method may also comprise actuating the at least one inlet within the at least one packer system to accept a fluid into the at least one inlet. The method may also comprise storing a portion of the fluid. The method may also comprise closing the at least one inlet and recovering the downhole apparatus.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the drawings. It is to be noted; however, that the appended drawings illustrate only typical embodiments of this disclosure and are; therefore, not be considered limiting of its scope, for the disclosure may admit to other equally effective embodiments.

FIG. 1 is a side view of a probe in one example embodiment of the disclosure.

FIG. 2 is a schematic of a flow layout for the example illustrated in FIG. 1.

FIG. 3 is a method for testing a formation with the probe of FIG. 1.

To facilitate understanding, identical reference numerals have been used, where possible, to designate identical elements that are common to the figures (“FIGS”). It is contemplated that elements disclosed in one embodiment may be beneficially utilized on other embodiments without specific recitation.

### DETAILED DESCRIPTION

In the following, reference is made to embodiments of the disclosure. It should be understood, however, that the disclosure is not limited to specific described embodiments. Instead, any combination of the following features and elements, whether related to different embodiments or not, is contemplated to implement and practice the disclosure. Furthermore, although embodiments of the disclosure may achieve advantages over other possible solutions and/or over the prior art, whether or not a particular advantage is achieved by a given embodiment is not limiting of the disclosure. Thus, the following aspects, features, embodiments and advantages are merely illustrative and are not considered elements or limitations of the claims except where explicitly recited in a claim. Likewise, reference to “the disclosure” shall not be construed as a generalization of inventive subject matter disclosed herein and should not be considered to be an element or limitation of the claims except where explicitly recited in a claim.

Although the terms first, second, third, etc., may be used herein to describe various elements, components, regions, layers and/or sections, these elements, components, regions, layers and/or sections should not be limited by these terms. These terms may be only used to distinguish one element, components, region, layer or section from another region, layer or section. Terms such as “first”, “second” and other numerical terms, when used herein, do not imply a sequence or order unless clearly indicated by the context. Thus, a first element, component, region, layer or section discussed herein could be termed a second element, component, region, layer or section without departing from the teachings of the example embodiments.

When an element or layer is referred to as being “on,” “engaged to,” “connected to,” or “coupled to” another element or layer, it may be directly on, engaged, connected, coupled to the other element or layer, or interleaving elements or layers may be present. In contrast, when an element is referred to as being “directly on,” “directly engaged to,” “directly connected to,” or “directly coupled to” another element or layer, there may be no interleaving elements or layers present. Other words used to describe the relationship between elements should be interpreted in a like fashion. As used herein, the term “and/or” includes any and all combinations of one or more of the associated listed terms.

Some embodiments will now be described with reference to the figures. Like elements in the various figures will be referenced with like numbers for consistency. In the following description, numerous details are set forth to provide an understanding of various embodiments and/or features. It will be understood, however, by those skilled in the art, that some embodiments may be practiced without many of these details, and that numerous variations or modifications from the described embodiments are possible. As used herein, the terms “above” and “below”, “up” and “down”, “upper” and “lower”, “upwardly” and “downwardly”, and other like terms indicating relative positions above or below a given point are used in this description to more clearly describe certain embodiments.

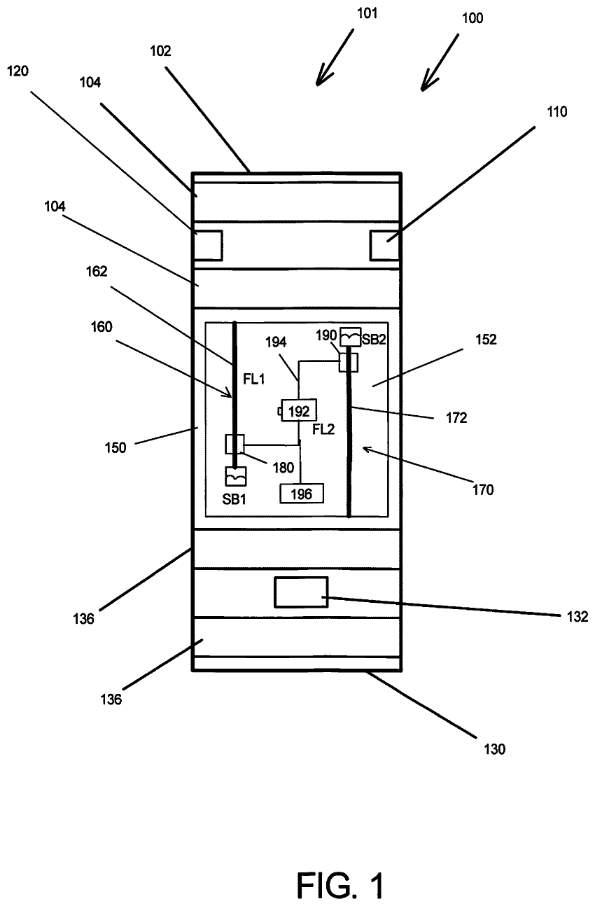

Referring to FIG. 1, a downhole focused probe/arrangement 100 is illustrated. The downhole focused probe/arrangement 100 (hereinafter “probe 100”) is configured to test formation fluids from a wellbore/subterranean environment. Aspects of the downhole focused probe 100 enable operators to sample formation fluids from narrow wellbores that are not achievable through conventional probes. Aspects of the downhole focused probe 100 may be used in environments that have “sticking” potential. Sticking refers to clay and silt materials that provide a cohesive suction to materials placed within the wellbore and may cause downhole tools to be semi-bonded within the wellbore. For purposes of definition, the term “wellbore” may include an open hole environment, a semi cased hole, a casing while drilling environment, or fully cased configuration. To remove the downhole tool, large pull forces and/or rotational forces are required. Aspects of the current description alleviate these concerns. In embodiments, the probe 100 is provided with a body 101 that houses components. The body 101 has a first end 102 and a second end 130.

At a first end 102 of the probe 100, a first inlet 110 and a second inlet 120 are provided for sampling formation fluids that may contain, for example, hydrocarbons from the subterranean environment. A first packer system 104 is provided to allow for the probe 100 to be set at the first end 102. In one embodiment, at the first end 102, the first inlet 110 and the second inlet 120 are provided at a 180 degrees orientation to one another. In another embodiment, the first inlet 110 and the second inlet 120 may be set together and independent of other inlets described later. For ease of description, the first inlet 110 and the second inlet 120 may be rotated 180 degrees compared to other inlets described later.

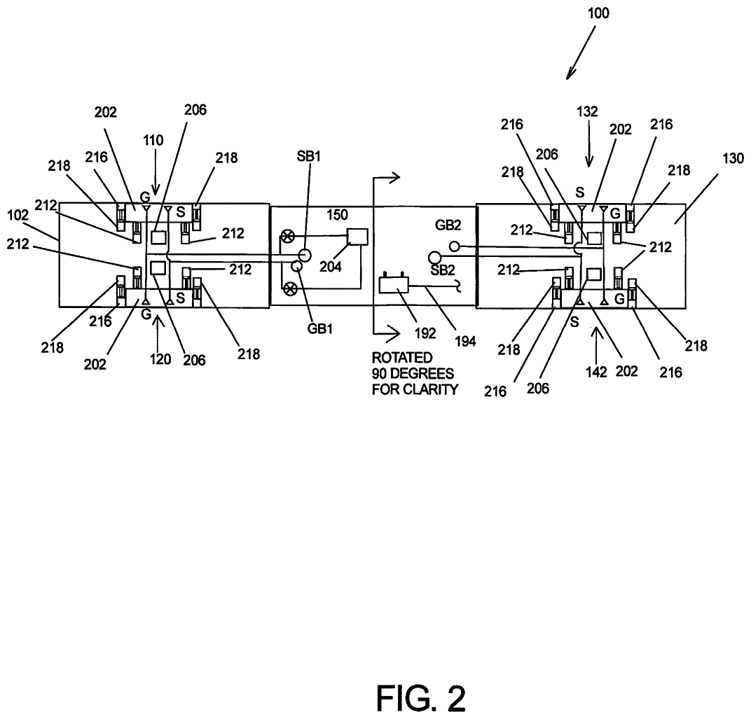

A second end 130 of the probe 100 provides a third inlet 132 and a fourth inlet (back side of drawing in FIG. 1 and shown in FIG. 2). The third inlet 132 and the fourth inlet 142 (shown in FIG. 2) may be rotated, in some embodiments, compared to the first inlet 110 and the second inlet 120. The rotation may be, for example, 90 degrees. Thus, in plain view, sampling may occur at 0 degrees, 90 degrees, 180 degrees and 270 degrees. The second end 130 of the probe 100 may have a second packer system 136 that allows the second end 130 to be set independently from the first end 102. Thus, either the first end 102 can be set, the first end 102 and the second end 130 can be set or just the second end 130 can be set in various configurations. Each of the first inlet 110 and second inlet 120 as well as the third inlet 132 and the fourth inlet 142 may be configured with a guard inlet (G) and a sample inlet (S). The guard inlet (G), in embodiments, may be located on an exterior periphery of the sample inlet (S) so that loosened materials from sampling may be intercepted by the guard inlet (G), allowing the sample inlet (S) to be free. Each of the guard inlets (G) and sample inlets (S) may be positioned within a probe block 214 (see, FIG. 2). The probe block 214 may be movable and actuated upon demand by operators, in non-limiting embodiments. In some embodiments, the probe 100 may be ruggedized wherein anchor plates 216 are installed around the probe block 214. The anchor plates 216 may be individually movement capable allowing the anchor plate 216 to be positioned next to the wellbore wall. In embodiments, hydraulic actuators may be provided for such positioning.

The first end 102 and second end 130 may be connected together through use of an interface block 150. The interface block 150 is configured to enclose a volume 152 where other equipment may be installed. Such equipment may be sample chambers/bottles, piping, solenoids, pumps, seals and valves. An example of such equipment is shown in corresponding FIG. 2.

In embodiments, two fluid systems 160, 170 are provided. A first fluid line (FL1) hereinafter fluid line 162 is provided for the first fluid system 160. A second fluid line (FL2) hereinafter fluid line 172 is provided for the second fluid system 170. Each of the fluid systems 160, 170 may have a separate sample bottle or sample bottles. Each of the fluid systems 160, 170 may be opened through a use of a set of solenoids 180, 190 that may be actuated through a battery 192 driven electrical system 194. An on-board computing arrangement 196 may actuate deployment of the packer 104, 136 systems, as discussed above, individually or together. The computing arrangement 196 may also be configured to operate individual solenoids 180, 190 to allow for selective sampling at each inlet for fluid in the probe 100. The computing arrangement 196 may also be configured to provide for actuation of other components, such as opening and closing of inlets. The computing arrangement 196 may be connected to a pump for allowing a draw on formation fluids. Fluid may be stored from the first fluid system 160 and second fluid system 170 in sample bottle SB1 and sample bottle SB2. Each of the sample bottles SB1, SB2 may be insulated and kept at desired temperature levels for use in analysis. Thus, the sample bottles SB1, SB2 may be heated and/or cooled, as necessary, operators. In embodiments, fluid analyzation may occur through an on-board analysis system, transferred to an attached fluid analyzation system, or as illustrated in FIG. 1, stored within the interior volume 152 for retrieval and use up-hole. The computing arrangement 196 may be configured to interface with a further testing system that may be incorporated into the probe 100 to allow for testing of fluids downhole and to provide communications with operators up-hole. The further testing system may include pressure, temperature, opacity and other quantities.

In one embodiment, the first end 102 and the second end 130 may act independently from the second end 130, as described above. Such ability to act independently allows for a complete redundancy of operations. Conventional operations do not have a “one drop” redundancy capability. In conventional operations, when a tool fails, the only possible solution is to retrieve the tool and place a new tool on a conveyance. Such time consuming and economically expensive alternatives are removed by providing a redundancy within a single tool. Such an advantage of redundancy is significant for deep wells and projects conveyed on drill pipe wherein removal of the tool from the hole can be problematic.

In embodiments where the four inlets are offset, problems that occur when all inlets are linear, are eliminated. In the linear probe alignments, one probe side is generally exposed to more effluent and materials than the other three. This results in plugging at one port and the sampling that occurs is compromised. By eliminating such close proximity and linearity of inlets, the plugging of one port is eliminated. The amount of offset of the first end 102 to the second end 130 allows the sampling to continue, uninterrupted. Such embodiments also protect the inner portions of the probe 100 as the inner seals are not compromised by materials during testing.

The embodiment of FIG. 1 also provides advantages that other conventional apparatus do not provide. Each packer system 104, 136 are generally smaller than other packer systems; therefore the packers systems 104, 136 do not require large amounts of time to inflate and seal the wellbore. Sealing of the packer systems 104, 136 allows for rapid deployment and testing, thereby saving economic expense. Additionally, since the packer systems 104, 136 do not require large amounts of fluid to inflate, space is saved within the overall tool for more vital components.

In non-limiting embodiments, aspects of the probe 100 allow for sampling and being able to freely move after sampling. Conventional apparatus, with inlets in a linear configuration, are prone to sticking at the rubber face and the wellbore due to the inability to equalize pressure if one inlet is blocked. In embodiments described herein; however, due to the offset nature of the inlets, sticking at the rubber faces does not occur.

In further non-limiting embodiments, the probe 100 is configured to resist the problem of bloating. In conventional arrangements, bloating occurs when a deformation occurs from overexpansion and/or clogging of the inlets. This leads to unequal expansion and possibilities of a tool stuck within the wellbore. Wear on the sealing surfaces of conventional arrangements is also a common problem due to friction contact with the wellbore. In the non-limiting embodiments described herein, the probe 100 has inlets that are independent. Thus, wear on a single surface will not impact the ability of the probe 100 from its intended function as two separate sampling ends are provided.

As a corollary to the above, deflation of the packers 104, 136 also takes less time than conventional apparatus. This allows for quicker removal of the probe 100 from the wellbore or moving to an alternative testing site. In conventional apparatus, deflation of the packer can take approximately seven minutes. Such times are significantly reduced by the packers 104, 136 of the probe 100.

Still further embodiments, allow for embodiments not possible in conventional apparatus. In one further embodiment, the first and second ends may be equipped to sample differing types of fluids. Conventional apparatus may be equipped to sample a single type of fluid, such as a water or an oil from a wellbore. In one example embodiment of the probe 100, the packer system 104 at the first end of the probe 100 may be equipped to sample water, while the packer system 136 at the second end of the probe 100 may be equipped to sample hydrocarbons. Thus, the individual packer systems 104, 136 and their associated inlets may be capable of different types of sampling. For example, inlets 132, 142 at the second end 130 may be configured with larger openings than the first end 102. Such larger inlets 132, 142 may be advantageous with sampling of some types of hydrocarbons. At the first end 102, smaller or more focused openings/inlets may be provided. According to wellbore engineers, the types of sampling inlets may be not only independent from one another, but may also have differing capabilities, greatly enhancing overall operational capability.

In still further embodiments, the probe 100 may be used in different formation types than conventional apparatus. In one embodiment, the inlets 110, 120, 132, 142 at the top and the bottom of the probe 100 may be configured with inlet surfaces that are greater in sampling percentage than conventional apparatus. Thus, in these embodiments, the probe 100 is configured to be used in tighter formations. In still further embodiments, larger packers 104, 136 may be used within a wellbore, thereby centering the probe 100 within the wellbore. Such centering may allow for more accurate readings by operators under some conditions. For example, packer systems with 8.5 inch or 10 inch may be used at the first end and the second end for either of the packer system 104, 136. Other configurations and sizes of packers may be used.

Referring to FIG. 2, schematic of internals of the probe 100 are illustrated. FIG. 2 illustrates the guard (G) and sample (S) lines more clearly. Each of the guard (G) lines may be fed to a centralized point within the interface block 150 and collected into a guard bottle (GB1). Similarly, each of the sample (S) lines may be fed to a centralized point within the interface block 150 and collected into a sample bottle (SB1). At the second end 130 a similar configuration to the first end 102 is provided. As will be understood, multiple sample bottles may be provided for both guard and sample inlet portions. Moreover, different sample bottles may be provided for the second end 130 and the first end 102, therefore operators may be able to determine what fluid was obtained from which end and which inlet.

A pump 204 may be used to create vacuum/draw on each of the inlets (guard or sample) 110, 120, 132, 142 at each of the ends 102, 130 of the probe 100. The pump 204 may have an associated fluid line with the pump 204 so that individual inlets may be chosen for creating a draw on the formation. Scrapers 206 may be provided at each inlet 110, 120, 132, 142 to allow for scraping of accumulated materials at the inlet to allow sampling to continue. The scraper 206 may be electrically operated through an electrical system 194 and associated battery 192. Probe deployment actuators 212 are provided at each probe block 214 to allow the probe block movement. Each of the probe deployment actuators 212 are individually movable. An anchor plate 216 is also positionable through a set of setting arrangements 218. Operators, therefore, may move either the anchor plate 216 or the probe block 214 a desired amount to allow for sampling to be conducted. As illustrated, the first fluid system 160 corresponds with the first end 102, while the second fluid system 170 corresponds to the second end 130. In embodiments, flow routing plugs and/or valves may be used in the interface block 150 without the need for a dual packer.

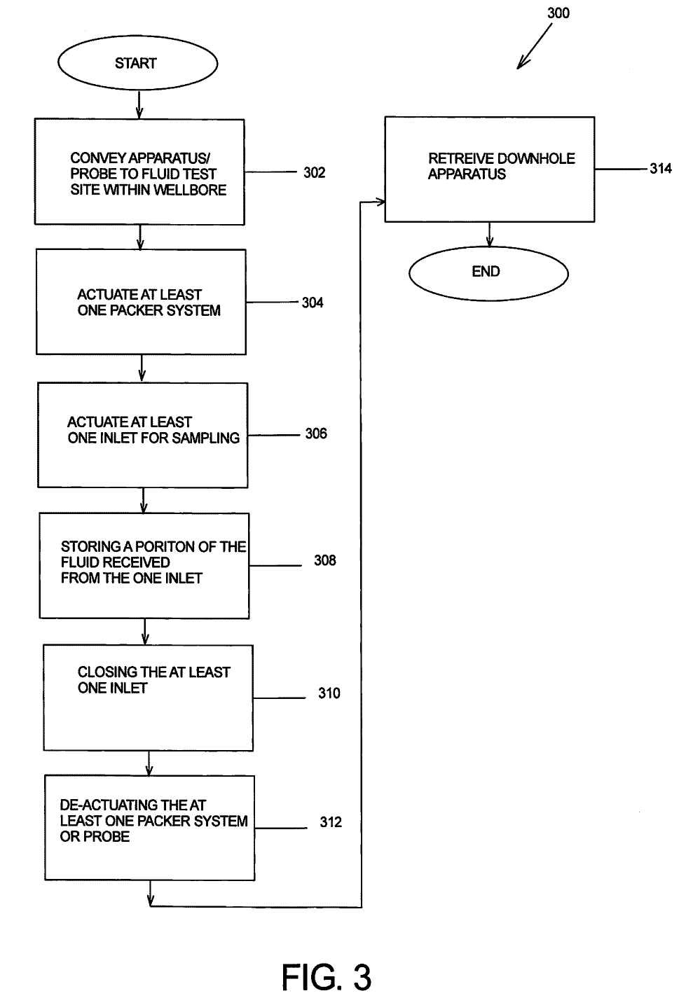

Referring to FIG. 3, a method 300 for sampling a fluid from a downhole environment is illustrated. The method may entail conveying a downhole apparatus to a location within a wellbore that is desired to be fluid tested at 302. An example downhole apparatus may include the probe 100 illustrated in FIG. 1. The conveyance of the downhole apparatus may be through a wireline conveyance or through drill pipe in non-limiting embodiments. At the position to be fluid tested within the wellbore, the method continues, at 304, with actuating at least one packer system in the downhole apparatus to achieve a tight seal between the at least one packer system and a wellbore wall, wherein the downhole apparatus is configured with two independent packer systems. As described above, a packer system may be installed at the first end 102 and at the second end 130. As will be understood, at 304, two individual packer systems may also be actuated. In some embodiments, individual packer systems may be deleted and a focused probe inlet only may be extended from the apparatus to abut to the wellbore wall. In such embodiments, the focused probe inlet may have an anchoring plate and/or probe block, as illustrated in FIG. 1. In embodiments using packer systems, the actuating may include filling up the packer systems with a fluid to achieve a leak tight seal. The filling up of the packer systems may occur through use of a pump that is located within the probe 100. In other embodiments, fluid may be pumped from the up-hole environment through the drill string, if so conveyed. After achieving the leak tight seal, either with extending a focused probe or through use of packers or both, at 306, the method may include actuating the at least one inlet within the leak tight seal of the actuated packer system. As will be understood, each end may have two individual inlets, as described above in relation to probe 100. The term actuating is defined as accepting fluid within the inlet so that the fluid may enter a fluid system within the probe. At 308, the method may further comprise storing a portion of fluid from a wellbore environment. At 310, the method may further comprise closing the at least one inlet. At 312, the method may further comprise de-actuating the at least one packer system in the downhole apparatus. At 314, the method may further comprise retrieving the downhole apparatus. As will be understood, more than one inlet may be actuated and sampled from during the method. Actuation of the independent ends may occur, at different times or simultaneously.

As will be understood, the method 300 as recited may be augmented by operators. The method 300 may also include steps of storing the fluid obtained through the inlets or the focused probe into individual sample bottles. The inlets themselves may be subdivided into both guard (G) and sample (S) inlets. In such embodiments, the guard inlets (G) may be located in a periphery to the sample inlet (S). Each end 102, 130 may incorporate inlets that are rotated compared to the opposite end. Each of the guard inlets (G) may be emptied into a separate sample bottle compared to each sample inlet (S). In such instances, operators may identify if the sampling of fluids is consistent across the entire inlet. In embodiments, additional pumps may be provided within the downhole apparatus so that pumping action may be taken for individual inlets. In embodiments where additional pumps are not used, a series of solenoid valves may be used to allow a single pump to create a pressure differential on the desired inlet. As provided above, a computing arrangement may control the solenoid valves according to the needs of operators. The computing arrangement, as described in FIG. 2, may be in contact with operators at surface level. This can be done through a wired connection, on one example embodiment. As the computing arrangement is in such contact, additional method steps of transmitting and receiving instructions to and from the surface and at the downhole apparatus level may be accomplished.

Embodiments of different potential arrangements and methods are described next. Such potential arrangements and methods are not to be considered limiting. In one embodiment, an arrangement is disclosed. The arrangement may comprise a body with a first end and a second end and an interface block. The arrangement may also comprise at least two first end inlets positioned in the first end and at least two second end inlets positioned in the second end, the first end inlets rotated related to the second end inlets. The arrangement may also comprise a first fluid system connected to the at least two first end inlets. The arrangement may also comprise a second fluid system connected to the at least two second end inlets. The arrangement may also comprise at least a first sample bottle connected to the first fluid end and at least a second sample bottle connected to the second fluid end, wherein the first fluid system is independent from the second fluid system.

In another example embodiment, the arrangement may further comprise a first packer system positioned around the first two end inlets and configured to establish a leak tight seal against a wellbore.

In another example embodiment, the arrangement may further comprise a second packer system positioned around the second end inlets and configured to establish a leak tight seal against the wellbore.

In another example embodiment, the arrangement may be configured wherein each of the first end inlets and second end inlets are configured with a selectively positionable probe block.

In another example embodiment, the arrangement may be configured wherein each of the first end inlets and second end inlets is further configured with a selectively positionable anchor plate.

In another example embodiment, the arrangement may be configured wherein the at least first sample bottle and second sample bottle are configured to keep liquid contents of the bottle at a specified temperature and pressure.

In another example embodiment, the arrangement may be configured wherein the at least two first end inlets positioned in the first end and the at least two second end inlets positioned in the second end are each configured with a guard inlet and a sample inlet.

In another example embodiment, the arrangement may be configured wherein each guard inlet is configured to accept fluid and collect fluid in a guard bottle.

In another example embodiment, the arrangement may be configured wherein each sample inlet is configured to accept fluid and collect fluid in a sample bottle.

In another example embodiment, the arrangement may be configured wherein the guard bottle is a first guard bottle and a second guard bottle and wherein fluid from the first end guard inlet is collected in a first guard bottle and fluid from the second end guard inlet is collected in a second guard bottle.

In another example embodiment, the arrangement may be configured wherein the sample bottle is a first sample bottle and a second sample bottle and wherein fluid from the first end sample inlet is collected in a first sample bottle and fluid from the second end sample inlet is collected in a second sample bottle.

In another example embodiment, the arrangement may further comprise a computing arrangement configured to transmit and receive signals to an up-hole environment.

In another example embodiment, the arrangement may further comprise at least one scraper configured to scrape material from at least one of the at least two first end inlets positioned in the first end and the at least two second end inlets.

In another example embodiment, a method for sampling fluids from a subterranean environment is disclosed. The method may comprise conveying a downhole apparatus to a location within a wellbore. The method may also comprise actuating at least one packer system in the downhole apparatus, the downhole apparatus having at least two inlets, each of the inlet rotated from the other inlet, wherein the downhole apparatus is configured with two independent packer systems. The method may also comprise actuating the at least one inlet within the at least one packer system to accept a fluid into the at least one inlet. The method may also comprise storing a portion of the fluid. The method may also comprise closing the at least one inlet and recovering the downhole apparatus.

In another example embodiment, the method may be performed wherein the downhole apparatus is conveyed through one of a wireline conveyance and drill pipe.

In another example embodiment, the method may be performed wherein the storing the portion of the fluid is within a sample bottle.

In another example embodiment, the method may be performed wherein the at least one inlet has a guard inlet and a sample inlet.

In another example embodiment, the method may be performed wherein the actuating the at least one inlet includes starting a pump to create a suction at the at least one inlet.

Description is provided related to measurements obtained during wireline operations generally performed. As will be understood, various changes and alterations may be accomplished during the attainment of the desired measurements and as such, methods described should not be considered limiting.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may be varied in many ways. Such variations are not to be regarded as a departure from the disclosure, and all such modifications are intended to be included within the scope of the disclosure.

While embodiments have been described herein, those skilled in the art, having benefit of this disclosure, will appreciate that other embodiments are envisioned that do not depart from the inventive scope. Accordingly, the scope of the present claims or any subsequent claims shall not be unduly limited by the description of the embodiments described herein.

What is claimed is:

1. An arrangement, comprising:
a body with a first end and a second end and an interface block;
at least two first end inlets positioned in the first end;
at least two second end inlets positioned in the second end, the first end inlets rotated related to the second end inlets;
a first fluid system connected to the at least two first end inlets;
a second fluid system connected to the at least two second end inlets;
at least a first sample bottle connected to the first fluid end; and
at least a second sample bottle connected to the second fluid end, wherein the first fluid system is independent from the second fluid system.

2. The arrangement according to claim 1, further comprising a first packer system positioned around the first two end inlets and configured to establish a leak tight seal against a wellbore.

3. The arrangement according to claim 2, further comprising a second packer system positioned around the second end inlets and configured to establish a leak tight seal against the wellbore.

4. The arrangement according to claim 1, wherein each of the first end inlets and second end inlets are configured with a selectively positionable probe block.

5. The arrangement according to claim 4, wherein each of the first end inlets and second end inlets is further configured with a selectively positionable anchor plate.

6. The arrangement according to claim 1, wherein the at least first sample bottle and second sample bottle are configured to keep liquid contents of the bottle at a specified temperature and pressure.

7. The arrangement according to claim 1, wherein the at least two first end inlets positioned in the first end and the at least two second end inlets positioned in the second end are each configured with a guard inlet and a sample inlet.

8. The arrangement according to claim 7, wherein each guard inlet is configured to accept fluid and collect fluid in a guard bottle.

9. The arrangement according to claim 7, wherein each sample inlet is configured to accept fluid and collect fluid in a sample bottle.

10. The arrangement according to claim 8, wherein the guard bottle is a first guard bottle and a second guard bottle and wherein fluid from the first end guard inlet is collected in a first guard bottle and fluid from the second end guard inlet is collected in a second guard bottle.

11. The arrangement according to claim 9, wherein the sample bottle is a first sample bottle and a second sample bottle and wherein fluid from the first end sample inlet is collected in a first sample bottle and fluid from the second end sample inlet is collected in a second sample bottle.

12. The arrangement according to claim 1, further comprising a computing arrangement configured to transmit and receive signals to an up-hole environment.

13. The arrangement according to claim 1, further comprising at least one scraper configured to scrape material from at least one of the at least two first end inlets positioned in the first end and the at least two second end inlets. positioned in the second end.

14. A method for sampling fluids from a subterranean environment, comprising:
conveying a downhole apparatus to a location within a wellbore;
actuating at least one packer system in the downhole apparatus, the downhole apparatus having at least two inlets, each of the inlets rotated from the other inlet, wherein the downhole apparatus is configured with two independent packer systems;
actuating at least one inlet within the at least one packer system to accept a fluid into the at least one inlet;
storing a portion of the fluid;
closing the at least one inlet; and
recovering the downhole apparatus.

15. The method according to claim 14, wherein the downhole apparatus is conveyed through one of a wireline conveyance and drill pipe.

16. The method according to claim 14, wherein the storing the portion of the fluid is within a sample bottle.

17. The method according to claim 14, wherein the at least one inlet has a guard inlet and a sample inlet.

18. The method according to claim 14, wherein the actuating the at least one inlet includes starting a pump to create a suction at the at least one inlet.

19. The method according to claim 14, wherein the closing of the at least one inlet is through actuation of a solenoid-controlled valve.

20. The method according to claim 14, wherein the recovering the downhole is through a wireline recovery.
